(19) United States
(12) Patent Application Publication
(10) Pub. No.: US 2024/0209348 A1
(43) Pub. Date: Jun. 27, 2024

(54) SOLID PHASE NEGATIVE ENRICHMENT

(71) Applicant: Harbinger Health, Inc., Cambridge, MA (US)

(72) Inventor: Anthony P. Shuber, Northbridge, MA (US)

(21) Appl. No.: 18/131,634

(22) Filed: Apr. 6, 2023

Related U.S. Application Data
(63) Continuation of application No. 16/179,523, filed on Nov. 2, 2018, now abandoned.
Application No. 16/179,523 is a continuation-in-part of application No. 16/018,926, filed on Jun. 26, 2018, now abandoned.
(60) Provisional application No. 62/672,217, filed on May 16, 2018.
(60) Provisional application No. 62/526,091, filed on Jun. 28, 2017.

(51) Int. Cl. C12N 15/10; C12Q 1/6806; C12Q 1/6886; G01N 27/447; G01N 30/88

(52) U.S. Cl. CPC C12N 15/1013; C12N 15/1003; C12Q 1/6806; C12Q 1/6886; G01N 27/44756; C12N 2310/20; C12Q 2600/156; G01N 2030/8827

(57) ABSTRACT

The invention provides methods for capturing target nucleic acid directly from bodily fluid samples, without the need for certain complex sample preparation steps, using Cas endonuclease to bind to the target nucleic acid sequences. The Cas proteins, along with their sequence-specific guide RNAs, may be introduced directly into the sample, where the Cas proteins bind to ends of a target nucleic acid. The target nucleic acid is thus isolated or enriched in a sequence-specific manner. The target nucleic acid may then be subject to any suitable detection or analysis assay, such as amplification or sequencing. The target nucleic acid may be enriched by digesting other, unbound nucleic acids present in the sample with exonuclease. The bound Cas proteins prevent exonuclease from digesting the target nucleic acid, thereby leaving the only the target nucleic acid substantially present in the sample.

### CROSS REFERENCE TO RELATED APPLICATIONS

This application is a continuation-in-part of U.S. Non-provisional application Ser. No. 16/018,926, filed Jun. 26, 2018, which claims the benefit of, and priority to, U.S. Provisional Application No. 62/526,091, filed Jun. 28, 2017 and U.S. Provisional Application No. 62/672,217, filed May 16, 2018, the contents of each of which are incorporated by reference.

### TECHNICAL FIELD

The invention relates to molecular genetics.

### BACKGROUND

When testing a patient for a disease, physicians obtain a biological sample from a patient, often times a bodily fluid, such as blood, saliva or a liquid biopsy, or a tissue sample, such as a tissue biopsy. After obtaining the biological sample from the patient, which may be a painful process for the patient, the sample must then be analyzed to determine the presence of the disease. Before even beginning the analysis of the sample to determine if the patient is suffering from a disease, the sample must be prepared so the nucleic acid can be sequenced. Existing preparation and analysis methods require expensive kits and reagents and time-consuming sample preparation procedures. Furthermore, some types of DNA, for example circulating tumor DNA, are present at low frequencies within cell-free DNA (cfDNA), and detection of such DNA is often difficult.

For example, in a liquid biopsy, a blood sample is taken from a patient and may be centrifuged to remove whole blood cells, leaving plasma or serum that includes the cfDNA. Typically, the sample must then be subjected to a sample preparation protocol before any genetic analysis is performed. For example, laboratory technicians use a commercially-available kit to aliquot the serum through a series of steps that require heating proteinase solutions to digest away proteins, lysis buffers to dissociate vesicles and other lipid fragments, and cleaning and suspension buffers. In some protocols, the resultant mixture is washed through a membrane within a column under vacuum after which the cfDNA is eluted from the column with a specialty wash buffer. This entire process requires hours or more and the use of expensive kits. With so many steps involved in a protocol, human error is inevitable. With error comes required retesting, which in turn further prolongs the receipt of the test, and thereby treatment. Often times physicians have prophylactically treated their patient because test results take too long. As such, patients are unnecessarily treated or treated improperly and effective treatment or adequate intervention is postponed until results are finally received by the physician.

Some companies offer specialty instruments to aid in automating some of those time-consuming and error prone steps, however, those instruments are not only expensive, but also require constant upkeep. Though the kits and instruments are expensive, they theoretically prepare and isolate, for example, cfDNA for analysis. Similar complex and time-consuming methodologies are used to prepare biological samples for other disease detection, such as infections. Time is of the essence for many patients suffering from certain diseases and by the time the result is provided to the physician, the disease or infection has progressed, sometimes past the point of intervention.

Furthermore, many preparation and sequencing techniques require a large biological sample in order to extract DNA and perform the sequencing. Large sample sizes mean more invasive and sometimes painful procedures for patients to endure.

In addition, using the next-generation sequencing (NGS) methodologies available today, studies have shown that up to 2% of the variants detected by NGS were not reproducible by Sanger Sequencing methodologies. Because of this many laboratories are required to confirm potential variants identified by NGS using Sanger Sequencing methodologies. Thereby, adding additional time onto the delivery of the test results to physicians and patients.

As such, there is a need in the field for a streamlined approach to prepare a biological sample for sequencing. Furthermore, an approach that can be utilized with any size sample or concentration of DNA would be advancement in the field. A need exists for improved sample preparation and detection so as to provide timely treatment to a patient.

### SUMMARY

The present invention provides methods for capturing target nucleic acid directly from biological samples without the need for significant sample preparation steps or kits. Methods of the invention capture a target nucleic acid from a biological sample. Methods of the invention utilize a complex of Cas, guide RNA and solid surface. Since target nucleic acid selectively binds to Cas endonuclease, when bound to the surface of a solid surface, or a particle Cas binds targets in the biological sample to the particle without the need for significant sample preparation. As such, Cas endonuclease when complexed with guide RNA on the particle, is used to bind the target nucleic acid of interest to the surface of the particle.

The Cas endonuclease is provided with one or more guide RNAs that bind to target nucleic acid that includes or flank a locus of interest, such as a locus of a known cancer-associated mutation or a specific genetic allele of clinical interest. The Cas endonuclease binds to and protects target nucleic acid even when a mutation is only present as a small fraction of the sample. When Cas endonuclease is bound to the surface of a particle, because the complex selectively binds the target nucleic acid to the surface of the particle, the described methods effectively isolate the target nucleic acid. Thus, methods of the invention are useful when analyzing nucleic acid present in low abundance in a sample such as blood or other bodily fluids. Once captured and processed, the target may then be analyzed or sequenced to report and use the genetic information, e.g., to detect or monitor disease, such as cancer.

In a preferred embodiment, Cas proteins, along with their sequence-specific guide RNAs (gRNA), are bound to a particle and the particle complex is introduced directly into a bodily fluid sample. The particle complex may be added to the collection tube prior to collection of the sample or added into collection tubes containing the sample. The gRNA mediates binding of the Cas proteins to a target nucleic acid of interest, such as tumor DNA fragment suspected of harboring a clinically significant variant.

The target nucleic acid may is enriched relative to other materials in the sample by any suitable enrichment methods, such as by elution of Cas proteins bound to the surface of the particle. The target nucleic acid may be enriched in the sample by elimination of non-target nucleic acid using, for example, exonucleases. Enrichment methods may be used alone or in combination with other enrichment methods. As a non-limiting example, exonuclease digestion may be used alone, or may be used before or after elution of the Cas proteins bound to the surface of the particle. The target nucleic acid may be subject to any suitable detection or analysis assay, such as electrophoresis, amplification or sequencing. Optionally, because the Cas proteins are bound to the surface of the particle, enrichment by elution and elimination of non-target nucleic acid is obtained directly on the surface of the particle. The target nucleic acid is enriched on the surface of the particle by elution by binding only the target nucleic acid to the Cas protein bound to the surface of the particle. Enrichment by elimination of non-target nucleic acid is obtained when the particle is removed from the sample. Once removed from the sample, the target nucleic acid may then be subject to any suitable detection or analysis assay, such as electrophoresis, amplification or sequencing.

Methods and related kits described herein are useful to detect the presence of a target nucleic acid, such as a variant, in a sample. Due to the nature by which a protein, such as a Cas complex, binds nucleic acid, methods may be used even where the target is present only in very small quantities, e.g., even as low as 0.01% frequency of mutant fragments among normal fragments in a sample (i.e., where about 50 copies of a circulating tumor DNA fragment harboring a mutation are present among about 500,000 unrelated fragments of similar size). Thus, methods of the invention may have particular applicability in discovering very rare, yet clinically important, information, such as mutations that are specific to a tumor and may be used to detect specific mutations among cell-free DNA, such as tumor mutations among circulating tumor DNA.

In a preferred method, CRISPR/Cas systems and associated guide RNAs are bound to a magnetic material and introduced to a bodily fluid sample. When used according to methods of the invention, Cas endonuclease, whether catalytically active or inactive, will bind to a target consistently via a guide RNA and will protect that target (i.e., stay bound) while remaining bound to the surface of the magnetic material, thereby allowing the target to be obtained out of the sample, either via elution of the magnetic material, or by elimination of non-target sequence. In certain other aspects of the invention, Cas endonuclease, whether catalytically active or inactive, will bind to a target consistently via a guide RNA and will protect that target (i.e., stay bound) while remaining bound to the surface of the magnetic material, thereby allowing the target to be enriched on the surface of the material by effectively extracting the target from the rest of the sample onto the surface and eliminating (excluding) non-target sequence from the surface of the material.

In certain aspects, the invention provides methods for detecting a target nucleic acid. Methods include obtaining a biological sample from a subject, introducing Cas proteins and guide RNA bound to the surface of a particle into the sample and binding the Cas proteins to ends of a target nucleic acid. The Cas protein may be a Cas endonuclease or catalytically deficient homolog thereof. The target nucleic may be both enriched or isolated from the sample.

The nucleic acid may be any naturally-occurring or artificial nucleic acid. The nucleic acid may be DNA, RNA, hybrid DNA/RNA, peptide nucleic acid (PNA), morpholino and locked nucleic acid (LNA), glycol nucleic acid (GNA), threose nucleic acid (TNA), or Xeno nucleic acid. The RNA may be a subpopulation of RNA, such as mRNA, tRNA, rRNA, miRNA, or siRNA. Preferably the nucleic acid is DNA.

The target or feature of interest may be any feature of a nucleic acid. The feature may be a variant. For example and without limitation, the feature may be a mutation, insertion, deletion, substitution, inversion, amplification, duplication, translocation, or polymorphism. The feature may be a nucleic acid from an infectious agent or pathogen. For example, the nucleic acid sample may be obtained from an organism, and the feature may contain a sequence foreign to the genome of that organism.

The target nucleic acid may be from a sub-population of nucleic acid within the nucleic acid sample. For example, the target nucleic acid may contain cell-free DNA, such as cell-free fetal DNA or circulating tumor DNA. In some embodiments, the sample includes plasma from the subject and the target nucleic acid is cell-free DNA (cfDNA). The plasma may be maternal plasma and the target may be of fetal DNA. In certain embodiments, the sample includes plasma from the subject and the target is circulating tumor DNA (ctDNA). In some embodiments, the sample includes at least one circulating tumor cell from a tumor and the target is tumor DNA from the tumor cell. In some embodiments, the target nucleic acid is complementary DNA (cDNA), which is made by reverse transcribing RNA. In some embodiments, detecting cDNA is a way to detecting target RNA.

The target nucleic acid may be from any source of nucleic acid. In preferred embodiments, the target nucleic acid is from a bodily fluid sample from a human. In preferred embodiments, the bodily fluid sample is a liquid or bodily fluid from a subject, such as bile, blood, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, vomit, or the like. In preferred embodiments, the bodily fluid sample is a blood sample, serum sample, plasma sample, urine sample, saliva sample, semen sample, feces sample, phlegm sample, or liquid biopsy. The sample may be a tissue sample from an animal, such as skin, conjunctiva, gastrointestinal tract, respiratory tract, vagina, placenta, uterus, oral cavity or nasal cavity. The sample may be a liquid biopsy or a tissue biopsy.

In some embodiments, obtaining the sample includes obtaining a bodily fluid sample from a subject in a collection tube. In a non-limiting example, the bodily fluid is blood and the collection tube is centrifuged to isolate serum or plasma from blood cells. The Cas endonuclease or catalytically deficient homolog thereof is bound to the surface of a particle and is introduced directly into the serum or plasma. In an embodiment, the Cas endonuclease or the catalytically deficient homolog thereof is bound to the surface of the particle is introduced into the serum or plasma as a ribonucleoprotein (RNP) in which the endonuclease is complexed with the guide RNA. Preferably, the guide RNA includes at least two single guide RNA molecules that each complex with a Cas endonuclease and guide the Cas endonuclease to hybridize to one of the targets, wherein the target includes a loci know to harbor a cancer-associated mutation. Even more preferably, the guide RNA is biotinylated. In an embodiment, the particle is comprised of magnetic, paramagnetic, or superparamagnetic material. Preferably, the particle is a magnetic bead and the surface of the bead is coated, wherein the surface coating is carboxyl, amino, hydroxyl, sulfates, tosyl, epoxy, chloromethyl, protein A, protein G, streptavidin, or biotin. In a preferred embodiment, the coating is streptavidin.

The method includes further separating the target nucleic acid bound to the particle complex from the sample. The method may include applying a magnetic field to the sample. The method further includes separating the target nucleic acid from the particle complex.

In other embodiments, the particle may include an agent that binds to a protein bound to an end of the target nucleic acid. The agent may an antibody or fragment thereof. The method may include chromatography, applying the sample to a column, or gel electrophoresis. The method may include further separating the protein-bound target nucleic acid from some or all of the unbound nucleic acid by size exclusion, ion exchange, or adsorption.

Each of the proteins may independently be any protein that binds a nucleic acid in a sequence-specific manner. The protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). The protein may be a catalytically inactive form of a nuclease, such as a programmable nuclease described above. The protein may be a transcription activator-like effector (TALE). The protein may be complexed with a nucleic acid that guides the protein to an end of the nucleic acid and is bound to a particle. For example, the protein may be a Cas endonuclease in a complex with one or more guide RNAs bound to a surface of a particle. Preferably, the protein is a Cas endonuclease or a catalytically deficient homolog thereof.

The target nucleic acid may be detected by any means known in the art. For example and without limitation, the target nucleic acid may be detected by DNA staining, spectrophotometry, sequencing, fluorescent probe hybridization, fluorescence resonance energy transfer, optical microscopy, or electron microscopy. Detecting the target nucleic acid may include identifying a variant in the target nucleic acid. Identifying the variant may include sequencing the nucleic acid (e.g., on a next-generation sequencing instrument), allele-specific amplification, and hybridizing a probe to the nucleic acid.

Methods of the invention may include amplifying the target nucleic acid to yield amplicons. Methods may further include sequencing the target nucleic acid to produce sequence reads and analyzing the sequence reads to provide genetic information of the subject or a pathogen. Methods may include analyzing the target nucleic acid to identify one or more variants in the subject.

In some embodiments, the target nucleic acid contains a variant specific to a disease. In other embodiments, the target nucleic acid contains a variant specific to cancer. The target nucleic acid may be present at no more than about 0.01% of cell-free DNA in the plasma or serum. By methods herein, the target nucleic acid is isolated and enriched from the serum or plasma.

In certain aspects, the invention provides a method for detecting a target nucleic acid. The method includes obtaining a serum or plasma sample from a subject, introducing Cas proteins and guide RNA bound to a surface of a particle into the serum or plasma, and binding the Cas proteins to ends of a target nucleic acid. The Cas protein may be a Cas endonuclease or a catalytically deficient homolog thereof. The guide RNA may be biotinylated. Unbound nucleic acid is digested from the sample by introducing exonuclease while the Cas proteins bound to the surface of the particle prevent the exonuclease from digesting the target nucleic acid, thereby enriching the sample for the target nucleic acid. The target nucleic acid complex may then be removed from the sample by using a separator, such as a magnetic field. Methods may include inactivating the exonuclease (e.g., by heating) prior to the removal step. Preferably, two Cas proteins bind to ends of the target nucleic acid and prevent the exonuclease from digesting the target nucleic acid. Methods may include unbinding the target nucleic acid from the particle by elution methods.

In other aspects, the invention provides a method for detecting a target nucleic acid. The method includes obtaining a biological sample from a subject, introducing Cas proteins and guide RNA bound to a surface of a particle into the sample, and binding the Cas proteins to ends of a target nucleic acid. The target nucleic acid is isolated from the sample on the surface of the particle and unbound nucleic acid does not bind to the surface, thereby enriching the surface with the target nucleic acid. The target nucleic acid complex may then be removed from the sample using a separator and the target nucleic acid may be unbound from the particle by elution methods.

Elution methods may include change of pH, change of ionic strength, polarity reducing agents (e.g., dioxane or ethyleneglycol), deforming eluents containing chaotropic salts, or affinity elution (e.g., elution of glycoproteins from lectin coated magnetic beads by the addition of free sugar).

The detecting step may include DNA staining, spectrophotometry, sequencing, fluorescent probe hybridization, fluorescence resonance energy transfer, optical microscopy, and electron microscopy. Certain methods may further include detecting the target nucleic acid (e.g., by amplification, sequencing, probe hybridization, digital PCR, etc.). Detecting the nucleic acid may include identifying a variant in the target nucleic acid. Identifying the variant may include sequencing the nucleic acid (e.g., on a next-generation sequencing instrument), allele-specific amplification, and hybridizing a probe the nucleic acid. Because the Cas proteins bound to a particle may be used to bind to the target nucleic acid in a sequence-specific manner, and thereby isolate and enrich for a specific variant, detecting the presence of the target nucleic acid may be useful to report the presence of the variant in a subject from whom the sample is obtained. In multiplexed embodiments, a panel or any number of specific mutations is assayed for through use of steps of the methods and the results may provide a description or count of tumor mutations detected from the target nucleic acid in the bodily fluid sample.

Furthermore, methods of the invention may include negative enrichment. As an example, Cas endonuclease may be provided with one or more guide RNAs bound to a particle that bind to a target nucleic acid and flank a loci of interest, such as a locus of a known cancer-associated mutation or a specific genetic allele of clinical interest. The Cas endonuclease bind to, and protect, mutation-containing nucleic acid even when the mutation is only present as a small fraction of the sample. The bound Cas proteins prevent exonuclease from digesting the target nucleic acid and, after incubation with exonuclease, the only nucleic acid substantially present in the sample is the target nucleic acid bound to the particle complex. Optionally, upon removal of the particle from the sample, the Cas proteins bound to the surface of the particle are the only nucleic acid present on the surface of the particle. The target nucleic acid is thus isolated or enriched in a sequence-specific manner. The target nucleic acid may then be subject to any suitable detection or analysis assay such as amplification or sequencing.

In a preferred method, CRISPR/Cas systems using guide RNAs specific for a mutation bound to a surface of a particle are introduced to the sample under conditions such that nucleic acid containing the variant is both bound to the surface of the particle and protected from exonuclease digestion while non-target nucleic acid in the sample is digested by exonuclease. When used according to methods of the invention, Cas endonuclease, whether catalytically active or inactive, will bind to a target consistently via a guide RNA and will protect (i.e., remain bound) that target nucleic acid bound to the surface of the particle for at least long enough that a promiscuous exonuclease can be reliably used to digest unbound, non-target nucleic acid from the sample. By protection of the isolated target bound to the surface of the particle with digestion of the non-target, a sample is effectively enriched for the target. Furthermore, by protection of the isolated target bound to the surface of the particle with digestion of the non-target nucleic acid, a target nucleic acid is effectively isolated from the sample and enriched for the target at the same time. As such, the target nucleic acid is thereafter successfully detected, captured, stored, isolated, preserved, sequenced, or otherwise assayed, all of which would be unobtainable without the methods of the present invention.

### BRIEF DESCRIPTION OF THE DRAWINGS

FIG. 1 shows a table of the inputs and the dilution amounts used in Example 1 described herein. Dilution 11 is at 3× concentration from previous experiments because the experiment uses 3× as much input DNA volume in the reaction. The copies per ul of stock, copies per ul in 50 ul reaction, amount of previous dilution (ul), plasma, and total volume (ul) are indicated.

FIG. 2 shows a table of the dilutions used in Example 1. For the percent of plasma in the final reaction, the percent of plasma in 2× sample, plasma dilution (ul), and tris dilution (ul) are shown in the table.

FIG. 3 shows a graph of the qPCR results after amplification from the post-cutting dilutions described in Example 1.

FIG. 4 shows the tabulated qPCR results from Example 1. Percent plasma, use of a Streck tube, amount of no Cas9 present, amount of Cas9 present, and percent cutting are indicated.

FIG. 5 shows a chart of the binding efficiency from Example 1, particularly showing the relationship between percent cleavage and percent plasma. In particular, the percent cleavage is shown as a function of the amount or percent of plasma in the cutting reaction. Results are shown for samples with no tube and samples using a Streck tube.

FIG. 6 shows a chart of the detection signal in plasma from Example 1, particularly showing the relationship between qPCR signal and percent plasma. In particular, the percent detection of no plasma in the sample is shown as a function of the percent plasma in the cutting reaction. Results are shown for samples with no tube and sample using a Streck tube.

FIG. 7 provides the reaction summary table of the formation of Cas9-guide RNA complexes for solid phase negative enrichment (e.g., magnetic bead) for use in samples comprised of 820 bp CFTR amplicon in plasma. 1.06 uL of the complex was used per bead-binding reaction.

FIG. 8 provides the composition of the control samples and the samples containing CFTR amplicon before adding 1.06 uL of the control or the Cas9 complex.

FIG. 9 provides an illustration of the reaction summary of the solid phase (i.e., streptavidin bead) bound Cas9 complex.

FIG. 10 depicts the results of an exemplary agarose gel electrophoresis of the activity of solid phase (e.g., streptavidin bead) Cas9 complexed with guide RNA CFTR F2 or biotinylated guide RNA CFTR F2 in a Tris solution containing 820 bp CFTR F2 target amplicon.

FIG. 11 depicts the results of an exemplary agarose gel electrophoresis of the activity of solid phase (e.g., streptavidin bead) Cas9 complexed with guide RNA CFTR F2 or biotinylated guide RNA CFTR F2 in plasma containing 820 bp CFTR F2 target amplicon.

### DETAILED DESCRIPTION

Methods of the invention provide for the enrichment of a target nucleic acid, in a sequence-specific manner, directly from bodily fluid samples without the need for complex sample preparation. Preferred embodiments include obtaining a bodily fluid sample from a subject. Certain embodiments of the invention provide a method for detecting a target nucleic acid in the bodily fluid sample.

Methods of the invention include introducing the Cas endonuclease, catalytically inactive Cas endonuclease, or homolog thereof and guide RNA into the bodily fluid sample. In a preferred embodiment, the binding proteins are provided by Cas endonuclease/guide RNA complexes. In yet another preferred embodiment, the binding proteins are provided by Cas endonuclease/guide RNA complexes bound to a particle. Embodiments of the invention use Cas endonuclease proteins that are originally encoded by genes that are associated with clustered regularly interspaced short palindromic repeats (CRISPR) in bacterial genomes. A CRISPR-associated (Cas) endonuclease may be introduced directly into the bodily fluid sample.

The Cas proteins bind to ends of a target nucleic acid. The target nucleic acid is thus isolated or enriched in a sequence-specific manner. The enriched target nucleic acid may then be subject to any suitable detection or analysis assay such as amplification or sequencing. The enriched target nucleic acid may be further enriched by digesting other, unbound nucleic acids present in the sample with exonuclease. The bound Cas proteins prevent the exonuclease from digesting the target nucleic acid, thereby leaving the only the target nucleic acid substantially present in the sample. The target nucleic acid is thus isolated or enriched in a sequence-specific manner. The target nucleic acid may then be subject to any suitable detection or analysis assay such as amplification or sequencing.

In another embodiment, the Cas proteins that are bound to a solid surface, such as a particle, bind to ends of a target nucleic acid. The target nucleic acid is thus isolated or enriched in a sequence-specific manner. The enriched target nucleic acid may then be subject to any suitable detection or analysis assay such as amplification or sequencing. The enriched target nucleic acid bound to the surface of the particle may be further enriched by digesting other, unbound nucleic acids present in the sample with exonuclease. The bound Cas proteins prevent the exonuclease from digesting the target nucleic acid, thereby leaving only the target nucleic acid bound to the particle substantially present in the sample. The target nucleic acid is thus isolated or enriched in a sequence-specific manner. The target nucleic acid may then be subject to any suitable detection or analysis assay such as amplification or sequencing.

Preferably, the Cas endonuclease is complexed with a guide RNA that targets the Cas endonuclease to a specific sequence. Any suitable Cas endonuclease or homolog thereof may be used. A Cas endonuclease (catalytically active or deactivated) may be Cas9 (e.g., spCas9), catalytically inactive Cas (dCas such as dCas9), Cpf1 (aka Cas12a), C2c2, Cas13, Cas13a, Cas13b, e.g., PsmCas13b, LbaCas13a, LwaCas13a, AsCas12a, others, modified variants thereof, and similar proteins or macromolecular complexes. The Cas13 proteins may be preferred where the target includes RNA. A Cas endonuclease/guide RNA complex includes a first Cas endonuclease and a first guide RNA. Preferably, the Cas endonuclease/guide RNA complex is bound to a particle. In the depicted embodiment, the complex comprises the Cas endonuclease or the catalytically deficient homolog thereof being introduced into the serum or plasma as a ribonucleoprotein (RNP) in which the Cas endonuclease or catalytically deficient homolog thereof is complexed with the guide RNA. The Cas endonuclease will bind to the target. The target may then be isolated or enriched, allowing for detection of the target.

The proteins that bind to ends of the target nucleic acid may be any proteins that bind to a nucleic acid in a sequence-specific manner. The protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). Programmable nucleases and their uses are described in, for example, Zhang, 2014, “CRISPR/Cas9 for genome editing: progress, implications and challenges”, Hum Mol Genet 23 (R1):R40-6; Ledford, 2016. CRISPR: gene editing is just the beginning, Nature. 531 (7593): 156-9; Hsu, 2014, Development and applications of CRISPR-Cas9 for genome engineering, Cell 157(6):1262-78; Boch, 2011, TALEs of genome targeting, Nat Biotech 29(2):135-6; Wood, 2011, Targeted genome editing across species using ZFNs and TALENs, Science 333(6040):307; Carroll, 2011, Genome engineering with zinc-finger nucleases, Genetics Soc Amer 188(4):773-782; and Urnov, 2010, Genome Editing with Engineered Zinc Finger Nucleases, Nat Rev Genet 11(9):636-646, each incorporated by reference.

The protein may be a catalytically inactive form of a nuclease, such as a programmable nuclease described above. The protein may be a transcription activator-like effector (TALE). The protein may be complexed with a nucleic acid that guides the protein to an end of the target nucleic acid. For example, the protein may be a Cas endonuclease in a complex with one or more guide RNAs. In another example, the protein may be a Cas endonuclease in a complex with one or more guide RNAs bound to a particle. In preferred embodiments, the protein is a Cas endonuclease, catalytically inactive Cas endonuclease, or homologs thereof.

In certain embodiments, the sample includes cfDNA from a subject. The sample is exposed to a first Cas endonuclease/guide RNA complex that binds to a target nucleic acid (e.g., a mutation of interest) in a sequence-specific fashion. In some embodiments, the sample is exposed to a first Cas endonuclease/guide RNA complex bound to a particle, that binds to a target nucleic acid (e.g., a mutation of interest) in a sequence-specific fashion. In some embodiments, the complex binds to a mutation in a sequence-specific manner. A segment of the nucleic acid, i.e., the target nucleic acid, is protected by introducing the first Cas endonuclease/guide RNA complex and a second Cas endonuclease/guide RNA complex that also binds to the nucleic acid. In preferred embodiments of the method, the guide RNA comprises at least two guide RNA molecules that each complex with a Cas endonuclease and guide the Cas endonuclease to hybridize to one target nucleic acid, wherein the target nucleic acid includes a loci know to harbor a cancer-associated mutation. In preferred embodiments of the method, the guide RNA comprises at least two guide RNA molecules that each complex with a Cas endonuclease bound to a particle and guide the Cas endonuclease to hybridize to one target nucleic acid, wherein the target nucleic acid includes a loci know to harbor a cancer-associated mutation.

Optionally, unprotected nucleic acid is digested. For example, one or more exonucleases may be introduced that promiscuously digest unbound, unprotected nucleic acid. Any suitable exonuclease may be used. Suitable exonucleases include, for example, Lambda exonuclease, RecJf, Exonuclease III, Exonuclease I, Exonuclease T, Exonuclease V, Exonuclease VII, T5 Exonuclease, and T7 Exonuclease, most of which are available from New England Biolabs (Ipswich, MA). While the exonucleases act, the target nucleic acid is protected by the bound complexes and survives the digestion step intact.

The described steps, including the digestion by the exonuclease leave a reaction product that includes principally only the mutant segment of nucleic acid, as well as any spent reagents, Cas endonuclease complexes, exonuclease, nucleotide monophosphates, and pyrophosphate as may be present. In certain embodiments, the described steps leave a reaction product that includes principally only the target nucleic acid bound to a particle, and spent reagents, Cas endonuclease complexes, exonuclease, nucleotide monophosphates, and pyrophosphate may also remain.

In certain embodiments, the exonuclease is deactivated. For example, exonuclease may be deactivated by following the manufacturer's instructions e.g., by heating to 90 degrees for a few minutes. After digestion, a positive selection step may be performed which may include, for example, amplification of the target nucleic acid by known methods or selection by an affinity assays.

The nucleic acid may be any naturally-occurring or artificial nucleic acid. The nucleic acid may be DNA, RNA, hybrid DNA/RNA, peptide nucleic acid (PNA), morpholino and locked nucleic acid (LNA), glycol nucleic acid (GNA), threose nucleic acid (TNA), or Xeno nucleic acid. The RNA may be a subpopulation of RNA, such as mRNA, tRNA, rRNA, miRNA, or siRNA. Preferably the nucleic acid is DNA.

The target or feature of interest may be any feature of a nucleic acid. The feature may be a variant, such as a mutation. The feature may be a mutation. For example and without limitation, the feature may be an insertion, deletion, substitution, inversion, amplification, duplication, translocation, or polymorphism. The feature may be a nucleic acid from an infectious agent or pathogen. For example, the nucleic acid sample may be obtained from an organism, and the feature may contain a sequence foreign to the genome of that organism.

The target nucleic acid may be from a sub-population of nucleic acid within the nucleic acid sample. For example, the target nucleic acid may contain cell-free DNA, such as cell-free fetal DNA or circulating tumor DNA. In some embodiments, the sample includes plasma from the subject and the target nucleic acid is cell-free DNA (cfDNA). The plasma may be maternal plasma and the target may be of fetal DNA. In certain embodiments, the sample includes plasma from the subject and the target is circulating tumor DNA (ctDNA). In some embodiments, the sample includes at least one circulating tumor cell from a tumor and the target is tumor DNA from the tumor cell. In some embodiments, the target nucleic acid is complementary DNA (cDNA), which is made by reverse transcribing RNA. In some embodiments, detecting cDNA is a way to detecting target RNA.

The target nucleic acid may be from any source of biological material. In preferred embodiments, the target nucleic acid is from a bodily fluid sample from a human. In preferred embodiments, the bodily fluid sample is a liquid or bodily fluid from a subject, such as bile, blood, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, vomit, or the like. In preferred embodiments, the bodily fluid sample is a blood sample, serum sample, plasma sample, urine sample, saliva sample, semen sample, feces sample, phlegm sample, or liquid biopsy. The sample may be a tissue sample from an animal, such as skin, conjunctiva, gastrointestinal tract, respiratory tract, vagina, placenta, uterus, oral cavity or nasal cavity. The sample may be a liquid biopsy or a tissue biopsy.

The method optionally includes detecting the target nucleic acid (which may harbor the mutation). Any suitable technique may be used to detect the target nucleic acid. For example, detection may be performed using DNA staining, spectrophotometry, sequencing, fluorescent probe hybridization, fluorescence resonance energy transfer, optical microscopy, electron microscopy, others, or combinations thereof. Detecting the target nucleic acid may indicate the presence of the mutation in the subject (i.e., a patient), and a report may be provided describing the mutation in the patient.

In an embodiment of the invention, a sample may contain a mutant fragment of DNA, a wild-type fragment of DNA, or both. A locus of interest is identified where a mutation may be present proximal to, or within, a protospacer adjacent motif (PAM). When the wild-type fragment is present, it may contain a wild-type allele at a homologous location in the fragment, also proximal to, or within, a PAM. A guide RNA is introduced to the sample that has a targeting portion complementary to the portion of the mutant fragment that includes the mutation. When a Cas endonuclease is introduced, it will form a complex with the guide RNA and bind to the mutant fragment but not to the wild-type fragment. The first Cas endonuclease/guide RNA complex includes a guide RNA with a targeting region that binds to the mutation but that does not bind to other variants at a loci of the mutation. In other embodiments, the Cas endonuclease and guide RNA are first bound to a particle. When the particle complex is introduced, the Cas endonuclease binds to the mutant fragment but not to the wild-type fragment. The described methodology may be used to target a mutation that is proximal to a PAM, or it may be used to target and detect a mutation in a PAM, e.g., a loss-of-PAM or gain-of-PAM mutation.

The described methodology may be used to target a mutation that is proximal to a PAM, or it may be used to target and detect a mutation in a PAM, e.g., a loss-of-PAM or gain-of-PAM mutation. The PAM is typically specific to, or defined by, the Cas endonuclease being used. For example, for Streptococcus pyogenes Cas9, the PAM includes NGG, and the targeted portion includes the 20 bases immediately 5′ to the PAM. As such, the targetable portion of the DNA includes any twenty-three consecutive bases that terminate in GG or that are mutated to terminate in GG. Such a pattern may be found to be distributed over ctDNA at such frequency that the potentially detectable mutations are abundant enough as to be representative of mutations over the tumor DNA at large. In such cases, mutation-specific enrichment may be used to detect mutations from a tumor. Moreover, methods may be used to determine a number of mutations over the representative, targetable portion of tumor DNA. Since the targetable portion of the genome is representative of the tumor DNA overall, the number of mutations may be used to infer a mutational burden for the tumor.

A feature of the method is that a specific mutation may be detected by a technique that includes detecting only the presence or absence of a fragment of DNA, and it need not be necessary to sequence DNA from a subject to describe mutations. Methods of the invention use protection at one or both ends of DNA segments. The gRNA selects for a known mutation on one end. A positive selection may be performed to positively select out the bound, target nucleic acid. In some embodiments, the bound target nucleic acid is bound to a particle and the particle is selected out of the sample. If the gRNA does not find the mutation, no protection is provided and the molecule may be digested, e.g. in negative enrichment, and the remaining molecules are either counted or sequenced. Optionally, after digestion, the particles are extracted and the bound target nucleic acid is quantified or sequenced. Methods are well suited for the analysis of samples in which the target of interest is extremely rare, and particularly for the analysis of maternal plasma or serum (e.g., for fetal DNA) or a liquid biopsy (e.g., for ctDNA).

Methods are useful for the isolation of intact DNA fragments of any arbitrary length and may preferably be used in some embodiments to isolate (or enrich for) arbitrarily long fragments of DNA, e.g., tens, hundreds, thousands, or tens of thousands of bases in length or longer. Long, isolated, intact fragments of DNA may be analyzed by any suitable method such as simple detection (e.g., via staining with ethidium bromide) or by single-molecule sequencing. It is noted that the Cas9/gRNA complexes may be subsequently or previously labeled using standard procedures. The complexes may be fluorescently labeled, e.g., with distinct fluorescent labels such that detecting involves detecting both labels together (e.g., after a dilution into fluid partitions). Preferred embodiments of the detection do not require PCR amplification and therefore significantly reduces cost and sequence bias associated with PCR amplification. Sample analysis can also be performed by a number of approaches, such as next generation sequencing (NGS), etc. However, many analytical platforms may require PCR amplification prior to analysis. Therefore, preferred embodiments of analysis of the reaction products include single molecule analysis that avoids the requirement of amplification.

Kits and methods of the invention are useful with methods disclosed in U.S. Provisional Patent Application 62/526,091, filed Jun. 28, 2017, for POLYNUCLEIC ACID MOLECULE ENRICHMENT METHODOLOGIES and U.S. Provisional Patent Application 62/519,051, filed Jun. 13, 2017, for POLYNUCLEIC ACID MOLECULE ENRICHMENT METHODOLOGIES, both incorporated by reference.

The target nucleic acid may be detected, sequenced, or counted. Where a plurality of fragments are present or expected, the fragment may be quantified, e.g., by qPCR.

The target nucleic acid may further be isolated or detected by any suitable method in order to separate the target segment from other nucleic acids in the sample. For example, the isolation or detection method may include separating the protein-bound target nucleic acid from some or all of the unbound nucleic acid. The isolation or detection method may include binding the protein-bound target nucleic acid to a particle. Optionally, the isolation method may include binding the target nucleic acid to a particle complex. The complex may include Cas and guide RNA bound to a particle. The particle may include magnetic, paramagnetic or superparamagnetic material. The isolation or detection method may include applying a magnetic field to the sample. The particle may include an agent that binds to a protein bound to an end of the target nucleic acid. The agent may an antibody or fragment thereof. The isolation or detection method may include chromatography. The isolation or detection method may include applying the sample to a column. The isolation or detection method may include separating the protein-bound target nucleic acid from some or all of the unbound nucleic acid by size exclusion, ion exchange, or adsorption. The isolation or detection method may include gel electrophoresis.

In a preferred embodiment, the isolation method includes binding the target nucleic acid to a particle complex. The complex may include Cas and guide RNA bound to a particle. The particle may include magnetic, paramagnetic or superparamagnetic material. Preferably, the particle is a magnetic bead and the surface of the bead is coated, wherein the surface coating is carboxyl, amino, hydroxyl, sulfates, tosyl, epoxy, chloromethyl, protein A, protein G, streptavidin, or biotin. In a preferred embodiment, the coating is streptavidin. The isolation or detection method may include applying a magnetic field to the sample. The isolation or detection method may be the presence of the target nucleic acid bound to the particle.

Embodiments of the invention may include detecting the target nucleic acid and optionally providing a report describing a mutation as present in the patient. The mutation-containing fragments may be detected by a suitable assay, such as sequencing, gel electrophoresis, a probe-based assay. The detection of the isolated segment of the target nucleic acid may be done by sequencing. The digestion provides a reaction product that includes principally only the target nucleic acid, as well as any spent reagents, Cas endonuclease complexes, exonuclease (e.g. when negative enrichment is performed), nucleotide monophosphates, or pyrophosphate as may be present. The reaction product may be provided as an aliquot (e.g., in a micro centrifuge tube such as that sold under the trademark EPPENDORF by Eppendorf North America (Hauppauge, NY) or glass cuvette). The reaction product aliquot may be disposed on a substrate. For example, the reaction product may be pipetted onto a glass slide and subsequently combed or dried to extend the fragment across the glass slide. The reaction product may optionally be amplified. Optionally, adaptors are ligated to ends of the reaction product, which adaptors may contain primer sites or sequencing adaptors. The presence of the segment in the reaction product aliquot may then be detected using an instrument.

The target nucleic acid may be detected by any means known in the art. For example and without limitation, the target nucleic acid may be detected by DNA staining, spectrophotometry, sequencing, fluorescent probe hybridization, fluorescence resonance energy transfer, optical microscopy, or electron microscopy. Detecting the nucleic acid may include identifying a variant in the nucleic acid. Identifying the variant may include sequencing the nucleic acid (e.g., on a next-generation sequencing instrument), allele-specific amplification, and hybridizing a probe to the nucleic acid. Methods of DNA sequencing are known in the art and described in, for example, Peterson, 2009, Generations of sequencing technologies, Genomics 93(2):105-11; Goodwin, 2016, Coming of age: ten years of next-generation sequencing technologies, Nat Rev Genet 17(6):333-51; and Morey, 2013, A glimpse into past, present, and future DNA sequencing, Mol Genet Metab 110(1-2):3-24, each incorporated by reference. Other methods of DNA detection are known in the art and described in, for example, Xu, 2014, Label-Free DNA Sequence Detection through FRET from a Fluorescent Polymer with Pyrene Excimer to SG, ACS Macro Lett 3(9):845-848, incorporated by reference.

One method for detection of protein-bound nucleic acids is immunomagnetic separation. Magnetic or paramagnetic particles are coated with an antibody that binds the protein bound to the segment, and a magnetic field is applied to separate particle-bound segment from other nucleic acids. In another embodiment, the protein-guide RNA complex is bound to the coated particles, and the target nucleic acid is protein bound. Methods of immunomagnetic purification of biological materials such as cells and macromolecules are known in the art and described in, for example, U.S. Pat. No. 8,318,445; Safarik and Safarikova, Magnetic techniques for the isolation and purification of proteins and peptides, Biomagn Res Technol. 2004; 2:7, doi: 10.1186/1477-044X-2-7, the contents of each of which are incorporated herein by reference. The antibody may be a full-length antibody, a fragment of an antibody, a naturally occurring antibody, a synthetic antibody, an engineered antibody, or a fragment of the aforementioned antibodies. Alternatively or additionally, the particles may be coated with another protein-binding moiety, such as an aptamer, peptide, receptor, ligand, or the like.

Chromatographic methods may be used for detection. In such methods, the bodily fluid sample is applied to a column, and the target nucleic acid is separated from other nucleic acids based on a difference in the properties of the target nucleic acid and the other nucleic acids. Size exclusion chromatography is useful for separating molecules based on differences in size and thus is useful when the segment is larger than other nucleic acids, for example the residual nucleic acids left from a digestion step. Methods of size exclusion chromatography are known in the art and described in, for example, Ballou, David P.; Benore, Marilee; Ninfa, Alexander J. (2008). Fundamental laboratory approaches for biochemistry and biotechnology (2nd ed.). Hoboken, N.J.: Wiley. p. 129. ISBN 9780470087664; Striegel, A. M.; and Kirkland, J. J.; Yau, W. W.; Bly, D. D.; Modern Size Exclusion Chromatography, Practice of Gel Permeation and Gel Filtration Chromatography, 2nd ed.; Wiley: NY, 2009, the contents of each of which are incorporated herein by reference.

Ion exchange chromatography uses an ion exchange mechanism to separate analytes based on their respective charges. Thus, ion exchange chromatography can be used with the proteins bound to the target nucleic acid impart a differential charge as compared to other nucleic acids. Methods of ion exchange chromatography are known in the art and described in, for example, Small, Hamish (1989). Ion chromatography. New York: Plenum Press. ISBN 0-306-43290-0; Tatjana Weiss, and Joachim Weiss (2005). Handbook of Ion Chromatography. Weinheim: Wiley-VCH. ISBN 3-527-28701-9; Gjerde, Douglas T.; Fritz, James S. (2000). Ion Chromatography. Weinheim: Wiley-VCH. ISBN 3-527-29914-9; and Jackson, Peter; Haddad, Paul R. (1990). Ion chromatography: principles and applications. Amsterdam: Elsevier. ISBN 0-444-88232-4, the contents of each of which are incorporated herein by reference.

Adsorption chromatography relies on difference in the ability of molecule to adsorb to a solid phase material. Larger nucleic acid molecules are more adsorbent on stationary phase surfaces than smaller nucleic acid molecules, so adsorption chromatography is useful when the target nucleic acid is larger than other nucleic acids, for example the residual nucleic acids left from a digestion step. Methods of adsorption chromatography are known in the art and described in, for example, Cady, 2003, Nucleic acid purification using microfabricated silicon structures. Biosensors and Bioelectronics, 19:59-66; Melzak, 1996, Driving Forces for DNA Adsorption to Silica in Perchlorate Solutions, J Colloid Interface Sci 181:635-644; Tian, 2000, Evaluation of Silica Resins for Direct and Efficient Extraction of DNA from Complex Biological Matrices in a Miniaturized Format, Anal Biochem 283:175-191; and Wolfe, 2002, Toward a microchip-based solid-phase extraction method for isolation of nucleic acids, Electrophoresis 23:727-733, each incorporated by reference.

Another method for detection is gel electrophoresis. Gel electrophoresis allows separation of molecules based on differences in their sizes and is thus useful when the target nucleic acid is larger than other nucleic acids, for example the residual nucleic acids left from a digestion step. Methods of gel electrophoresis are known in the art and described in, for example, Tom Maniatis; E. F. Fritsch; Joseph Sambrook. “Chapter 5, protocol 1”. Molecular Cloning-A Laboratory Manual. 1 (3rd ed.). p. 5.2-5.3. ISBN 978-0879691363; and Ninfa, Alexander J.; Ballou, David P.; Benore, Marilee (2009). fundamental laboratory approaches for biochemistry and biotechnology. Hoboken, NJ: Wiley. p. 161. ISBN 0470087668, the contents of which are incorporated herein by reference.

Certain preferred embodiments include obtaining a blood, plasma, or serum sample from a patient. The blood, plasma, or serum may include cfDNA and thus also include ctDNA among the cfDNA. Specific sequences of the ctDNA are isolated or enriched and analyzed or detected to detect or report genetic information from the subject, such as a presence or count of certain tumor mutations. Methods of the invention include introducing Cas endonucleases (or catalytically inactive homologs thereof such as dCas9) directly into serum or plasma. The Cas endonucleases are complexed with guide RNAs that include targeting portions specific for a target nucleic acid. Methods of the invention also include introducing Cas endonucleases complexed with guide RNAs that include targeting portions specific for a target nucleic acid, bound to the surface of a particle, directly into plasma or serum. In the plasma or serum, the complexes bind to ends of the target and protect it. Exonuclease may be introduced to digest unbound nucleic acid into monomers and fragments too small for further meaningful detection, sequencing, or amplification.

Embodiments of the invention provide for treatment of a sample. For example, a blood sample may be obtained from a patient. The sample may be collected in any suitable blood collection tube such as the collection tube sold under the trademark VACUTAINER by BD (Franklin Lakes, NJ). In certain embodiments, the collection tube comprises an EDTA collection tube, and Na-EDTA collection tube or the collection tube sold under the trademark CELL-FREE DNA BCT by Streck, Inc. (La Vista, NE), sometimes referred to in the art as a Streck tube. Use of a Streck tube stabilizes nucleated blood cells and prevents the release of genomic DNA into the sample. This facilitates the collection of sample that includes cell-free DNA.

The sample may be centrifuged to generate a sample that includes a pellet of blood cells and a supernatant, which contains serum or plasma. Serum is the liquid supernatant of whole blood that is collected after the blood is allowed to clot and centrifuged. Plasma is produced when the process includes an anticoagulant. To collect serum, blood is collected in tubes. After collection, the blood is allowed to clot by leaving it undisturbed at room temperature (about 15-30 minutes). The clot is removed by centrifuging, e.g., at 1,000-2,000×g for 10 minutes in a refrigerated centrifuge. The resulting supernatant is designated serum and may be transferred to a clean polypropylene tube using a Pasteur pipette. For plasma, blood is collected into commercially available anticoagulant-treated tubes e.g., EDTA-treated (lavender tops), citrate-treated (light blue tops), or heparinized tubes (green tops), followed by centrifugation to collect the supernatant. The supernatant is preferably transferred to a fresh tube, away from the pellet, which may be discarded. Particularly where the collection tube included an anticoagulant, the transfer should give a good separation of the plasma from the whole blood cells. After transfer, the sample includes plasma or serum, which includes cfDNA.

In an exemplary embodiment, serum or plasma is transferred from a centrifuge tube to a new tube, complexes comprising Cas9 and guide RNA are added, and the mixture is incubated. For example, amplification or an affinity assay may be performed to positively select out the bound, target nucleic acid. In another embodiment, exonuclease may be introduced to digest unbound, non-target DNA, and then the exonuclease may be deactivated (e.g., by heat). A positive selection may then follow (e.g., amplification or an affinity assay) to positively select out the bound, target nucleic acid.

In another exemplary embodiment, serum or plasma is transferred from a centrifuge tube to a new tube, complexes comprising Cas9 and guide RNA bound to a particle are added, and the mixture is incubated. For example, positive selection of the target nucleic is performed when the target nucleic acid binds to the complex, and can then be further eluted from the particle. In another embodiment, exonuclease may be introduced to digest unbound, non-target DNA in the sample, and then the exonuclease may be deactivated (e.g., by heat). A positive selection may then follow (e.g., elution by magnetic stand) to positively select out the bound, target nucleic acid.

In another exemplary embodiment, plasma or serum is removed from the centrifuge tube (the supernatant) and transferred into a new tube. Appropriate buffers/reagents are added to modify a chemical environment to promote binding of Cas endonuclease to the target nucleic acid. For example, pH can be adjusted, as may temperature, salinity, or co-factors present. The Cas complexes are added and allowed to incubate. For example, amplification or an affinity may be performed to positively select out the bound, target nucleic acid. An exonuclease may optionally be added, which ablates all free, non-target nucleic acid. The target may be positively selected such as by amplification or an affinity assay after exonuclease digestion of the non-target nucleic acid.

In another exemplary embodiment, a guide RNA is incubated with a Cas nuclease. In an embodiment, the guide RNA is biotinylated. The Cas nuclease guide RNA complex is then incubated with a particle to form a Cas-gRNA particle complex. In preferred embodiment, the particle is a magnetic bead. The plasma or serum is removed from the centrifuge tube (the supernatant) and transferred into a new tube. Appropriate buffers/reagents are added to modify a chemical environment to promote binding of the target nucleic acid to the Cas-gRNA particle complex. For example, pH can be adjusted, as may temperature, salinity, or co-factors present. The Cas-gRNA particle complexes are added and allowed to incubate. For example, positive selection of the target nucleic occurs when the target nucleic acid binds to the Cas-gRNA particle complex. An exonuclease may optionally be added, which ablates all free, non-target nucleic acid. In another example, a magnetic stand isolates the beads from the sample. The target may be positively selected such as by a magnetic stand after exonuclease digestion of the non-target nucleic acid.

Methods may include detection or isolation of circulating tumor cells (CTCs) from a blood sample. Cytometric approaches use immunostaining profiles to identify CTCs. CTC methods may employ an enrichment step to optimize the probability of rare cell detection, achievable through immune-magnetic separation, centrifugation, or filtration. Cytometric CTC technology includes the CTC analysis platform sold under the trademark CELLSEARCH by Veridex LLC (Huntingdon Valley, PA). Such systems provide semi-automation and proven reproducibility, reliability, sensitivity, linearity and accuracy. See Krebs, 2010, Circulating tumor cells, Ther Adv Med Oncol 2(6):351-365 and Miller, 2010, Significance of circulating tumor cells detected by the CellSearch system in patients with metastatic breast colorectal and prostate cancer, J Oncol 2010:617421-617421, both incorporated by reference.

Certain embodiments of the invention may provide a kit. The kit preferably includes reagents and materials useful for performing methods of the invention. For example, the kit may include one or more guide RNA that, taken in pairs, are designed to flank cancer-associated mutations. The kit may include one or more biotinylated guide RNAs that, taken in pairs, are designed to flank cancer-associated mutations. The kit may include one or more guide RNAs that are mutation specific and only hybridize to target that includes a mutation. The kit may include one or more biotinylated guide RNAs that are mutation specific and only hybridize to target that includes a mutation. The kit may include a Cas endonuclease or a nucleic acid encoding a Cas endonuclease such as a plasmid. The kit may optionally include exonuclease. The kit may include a particle for the protein-guide RNA complex to bind. The kit may include a magnetic patricle. The kit may include a coated magnetic bead. The kit may include a streptavidin magnetic bead. The kit may include reagents for adjusting conditions such as pH, salinity, co-factors, etc., to promote binding or activity of Cas endonuclease (including to promote binding of catalytically inactive Cas endonuclease, which may be included as the Cas endonuclease) in the bodily fluid sample, such as plasma or serum. The kit may further include instructional materials for performing methods of the invention, and components of the kit may be packaged in a box suitable for shipping or storage. Preferably, the kit contains one or more collection tubes, such as a blood collection tube.

The Cas endonuclease/guide RNA complexes can be designed to bind to variants or specifically mutations of clinical significance, such as a mutation specific to a tumor. When a such a variant is thus detected, a report may be provided to, for example, describe the mutation and the disease in a patient or a subject. Thus, certain embodiments may comprise providing a report. The report preferably includes a description of the mutation in the subject (e.g., a patient). The report includes a description of the disease the subject (e.g., a patient) is suffering from. The method for detecting rare nucleic acid may be used in conjunction with a method of describing variants or mutations (e.g., as described herein). Either or both detection processes may be performed over any number of loci in a patient's genome or preferably in a patient's tumor DNA. As such, the report may include a description of a plurality of variants (e.g., structural alterations or mutations) in the patient's genome or tumor DNA, or the pathogen's DNA. As such, the report may give a description of a mutational landscape of a tumor.

Knowledge of a mutational landscape of a tumor may be used to inform treatment decisions, monitor therapy, detect remissions, or combinations thereof. For example, where the report includes a description of a plurality of mutations, the report may also include an estimate of a tumor mutation burden (TMB) for a tumor. It may be found that TMB is predictive of success of immunotherapy in treating a tumor, and thus methods described herein may be used for treating a tumor.

Knowledge of the presence of a disease may be used to inform treatment decisions. For example, the report might describe a variant associated with a disease and describe the treatment associated with the disease. Thus, methods of this invention may be used to treat a disease.

Methods of the invention may be used to detect and report clinically actionable information about a patient, a pathogen present in a patient, or a tumor in a patient. For example, the method may be used to provide a report describing the presence of the genomic alteration in a genome of a subject. Additionally, protecting a segment of DNA, and optionally digesting unprotected DNA, provides a method for isolation or enrichment of DNA fragments, i.e., the protected segment. It may be found that the described enrichment techniques are well-suited to the isolation/enrichment of arbitrarily long DNA fragments, e.g., thousands to tens of thousands of bases in length or longer.

Long DNA fragment targeted enrichment, or negative enrichment, creates the opportunity of applying long read platforms in clinical diagnostics. Negative enrichment may be used to enrich “representative” genomic regions that can allow an investigator to identify “off rate” when performing CRISPR Cas9 experimentation, as well as enrich for genomic regions that would be used to determine TMB for immuno-oncology associated therapeutic treatments. In such applications, the negative enrichment technology is utilized to enrich large regions (>50 kb) within the genome of interest.

By the described methods, a bodily fluid sample can be assayed for a mutation using a technique that is inexpensive, quick, and reliable. Methods of the invention are conducive to high throughput embodiments, and may be performed, for example, in droplets on a microfluidic device, to rapidly assay a large number of aliquots from a sample for one or any number of genomic structural alterations.

### Example 1

The cutting efficiency of amplicons by Cas9 in plasma is shown by experiment. Results from the experiment indicated that Cas proteins bind to expected cognate targets under guide RNA guidance in plasma or serum. In particular, Cas9 was tested for cutting activity in plasma in an experimental protocol.

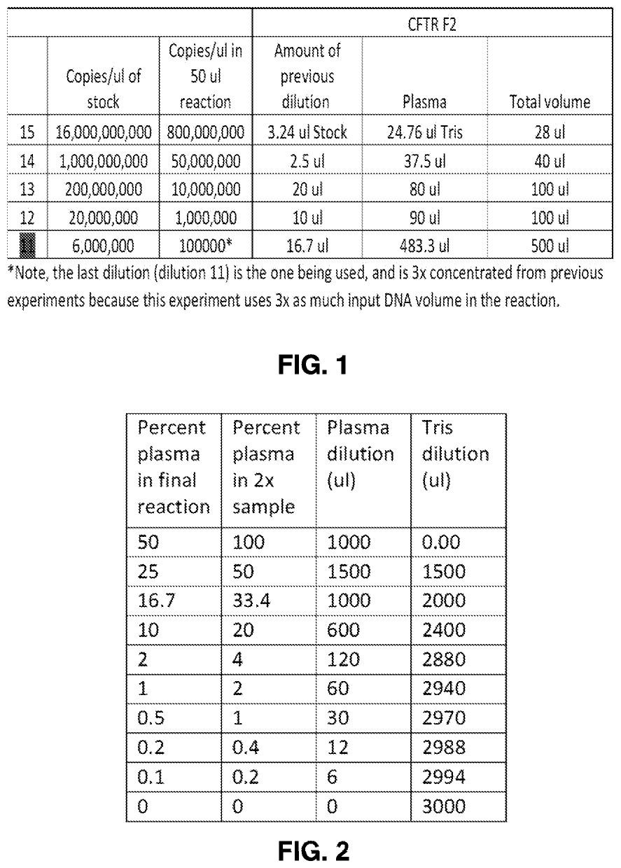

Plasma samples were placed in Streck tubes and in standard tubes. The experiments used an 800 bp amplicon from the cystic fibrosis transmembrane receptor gene. Dilutions were made of CFTR F2 800 bp into plasma with 5 million copies per reaction total (FIG. 1). The percent plasma in reaction after dilution was 50%, 25%, 16.7%, 10%, 2%, 1%, 0.5%, 0.2%, 0.1%, and 0% (FIG. 2).

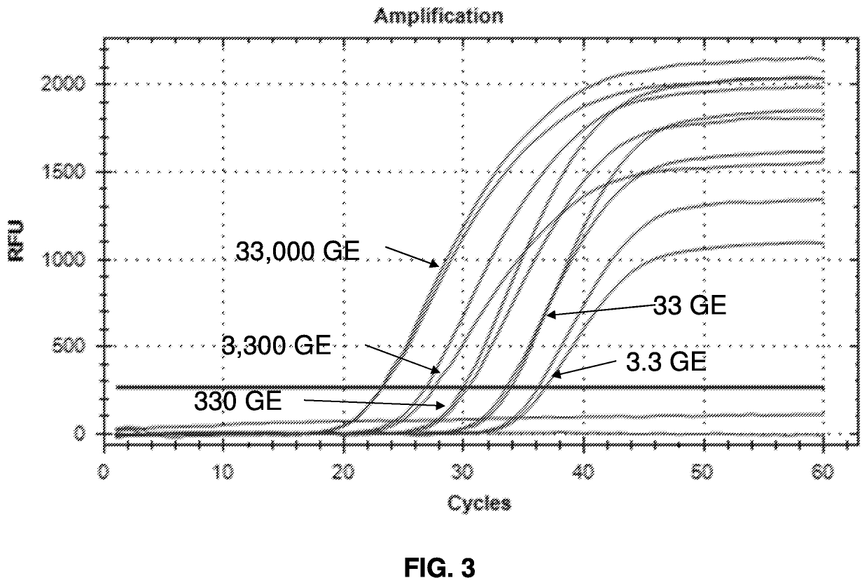

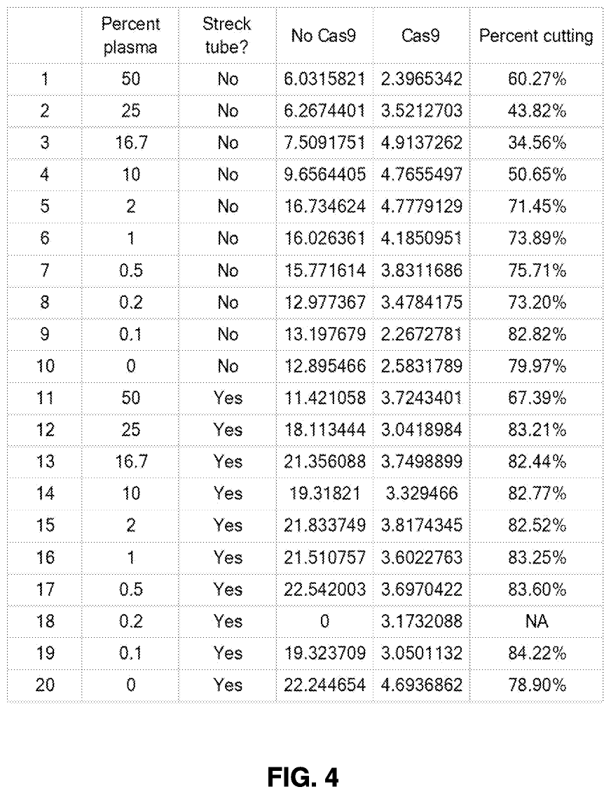

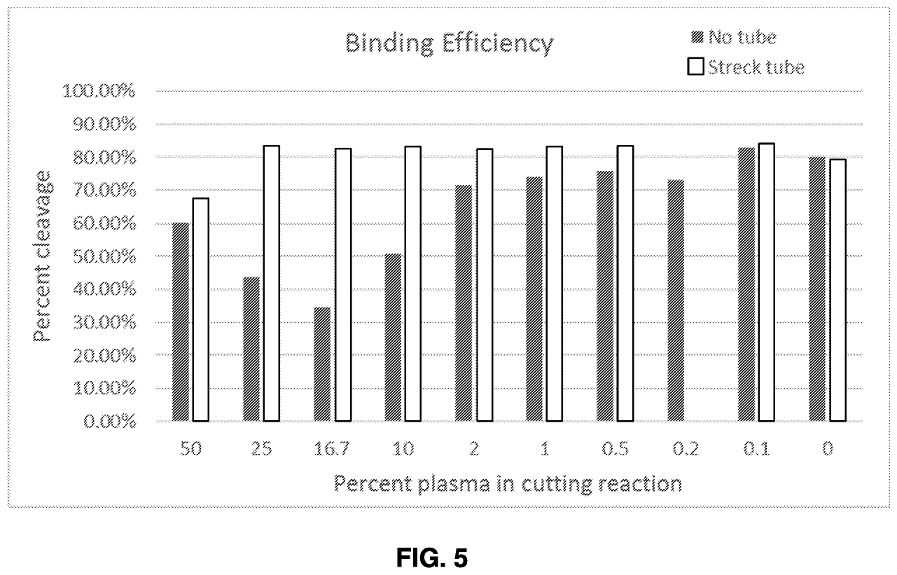

Cas9 with guide RNA was added and allowed to cut. qPCR was then used to probe across the cut site. For qPCR, samples were diluted 1/100, and then 5 ul were used per 20 ul reaction. The qPCR results were analyzed from amplifying, post-cutting, from dilutions (FIGS. 3 and 4). The qPCR results indicated cleavage as a function of plasma amount (FIG. 5). For example, every replicate in a Streck tube demonstrated greater than 60% cutting efficiency by Cas9 in the CFTR amplicon. Cas9 exhibited detectable cutting, even in standard, non-Streck tubes.

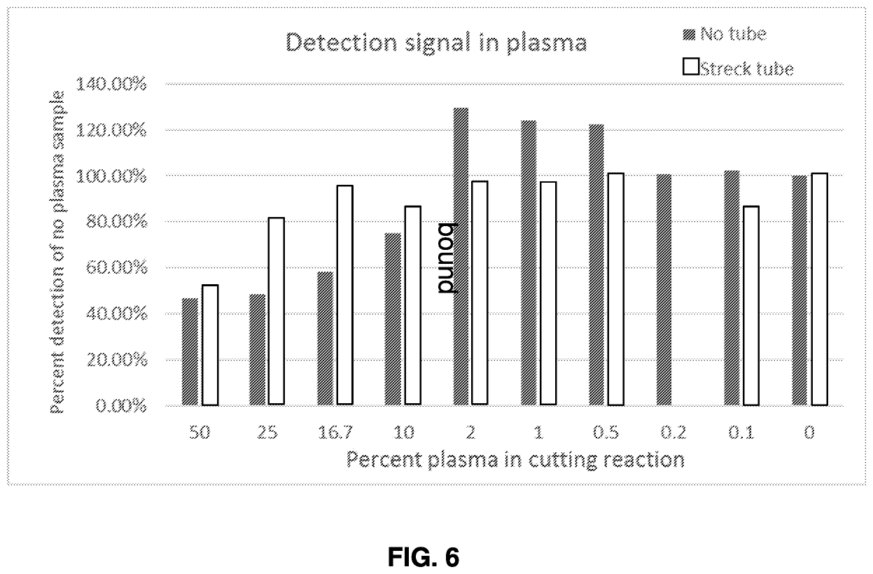

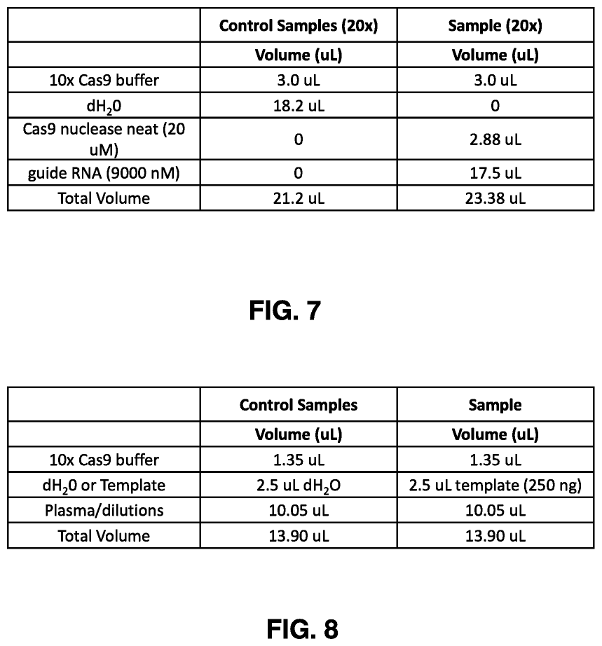

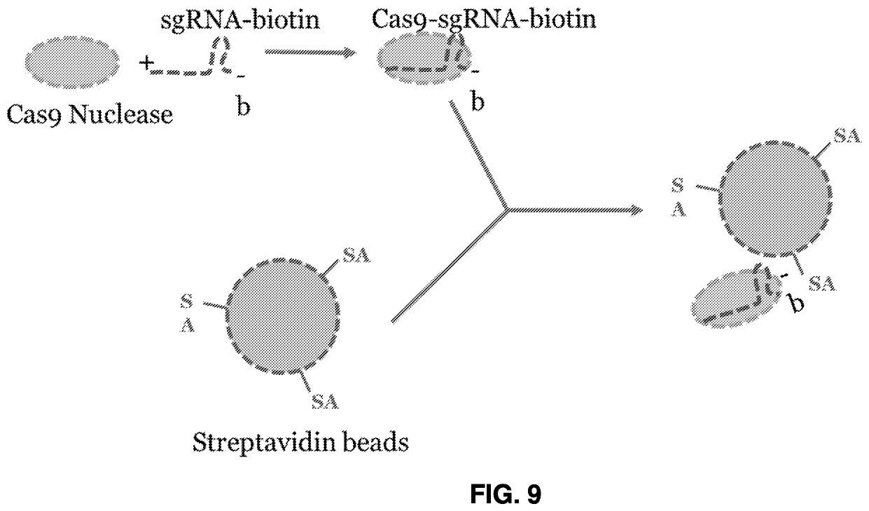

The results also indicated a relationship between the qPCR signal and percent plasma (FIG. 6). For example, the data show Cas9 exhibits detectable cutting in Na-EDTA plasma. For the reactions performed in straight plasma, cutting efficiency in 2% plasma or lower resembled no plasma cutting efficiency (82.82% for in plasma compared to 79.97% in no plasma). For the reactions performed in plasma incubated in a Streck tube, the cutting efficiency in 25% plasma or lower resembled no-plasma cutting efficiency (83.14% compared to 78.90%). Further, there was 60-67% cutting for the 50% plasma samples. In 50% plasma, CRISPR/Cas9 complexes retained 75% activity. Results of the data show that Cas endonuclease and homologs thereof bind to target DNA under guidance of guide RNA in plasma.

### Example 2

The cutting and protection activity of Cas9 on a solid surface is shown in this experiment. Results from this experiment indicate that a complex of Cas9-guide RNA and a particle, in this example, a magnetic bead coated with streptavidin, directly inserted into plasma or serum, obtain negative enrichment. That is, the magnetic bead bound Cas proteins bind to expected targets under guide RNA guidance in plasma or serum and cut and protect the target nucleic acid to achieve solid phase negative enrichment.

This experiment utilized samples comprised of 820 bp amplicon from the cystic fibrosis transmembrane receptor gene spiked into human plasma. Samples were made consisting of the 820 bp CFTR amplicon added to 10 mM Tris (250 ng, 2.5 uL, 100 ng/uL), pH 7.5 or human plasma. The Cas9-guide RNA complexes were made consisting of: 3.0 uL of Cas9 buffer (10×), 2.88 uL of Cas9 (20 uM) complexed with 17.5 uL of CFTR F2 guide RNA (9000 nM) (FIG. 1). The control “complexes” made consisted of 18.2 uL of water and 3.0 uL of 10× Cas9 buffer. 1.06 uL of the Cas9-guide RNA reagents were incubated (45 minutes; 25° C.) with 25 uL Dynabeads M-289 beads and then washed two times in Cas9 buffer.

Next, the bead bound Cas9-RNA complexes were resuspended in either the human plasma in a Streck tube or the Tris buffer solution. The samples were then incubated (60 minutes; 37 C), which allowed for the Cas9 to bind and cleave the target nucleic acid.

After the cleavage reaction, half of the samples were incubated (1 hour, 37 C) with lambda exonuclease (15 units) and exonuclease VII (15 units) to degrade any unprotected DNA. The bead complexes were then placed on a magnetic stand and the supernatant fraction was collected. After washing the beads in Cas9 buffer, the beads were resuspended in Cas9 buffer. The samples were then phenol-chloroform extracted, ethanol precipitated (with 10 ug glycogen added), and re-suspended in 10 mM Tris, pH 7.5, to achieve a final volume of 30 uL. Agarose gel electrophoresis (with a volume representing 125 ng equivalent starting sample) was used for analysis.

The results indicate solid phase negative enrichment directly in a bodily fluid sample is possible using a Cas9-guide RNA complex that binds to and protects a target nucleic acid sequence on a solid surface, such as a bead. That is, treating an amplicon DNA with Cas9 complexed with either guide RNA CFTR F2 or biotinylated guide RNA CFTR F2 bound to a magnetic bead, Cas9 is able to both cut and protect the target nucleic on the surface of the bead.

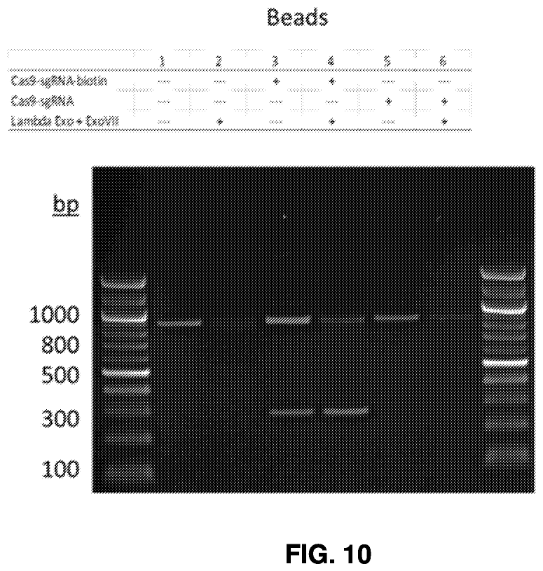

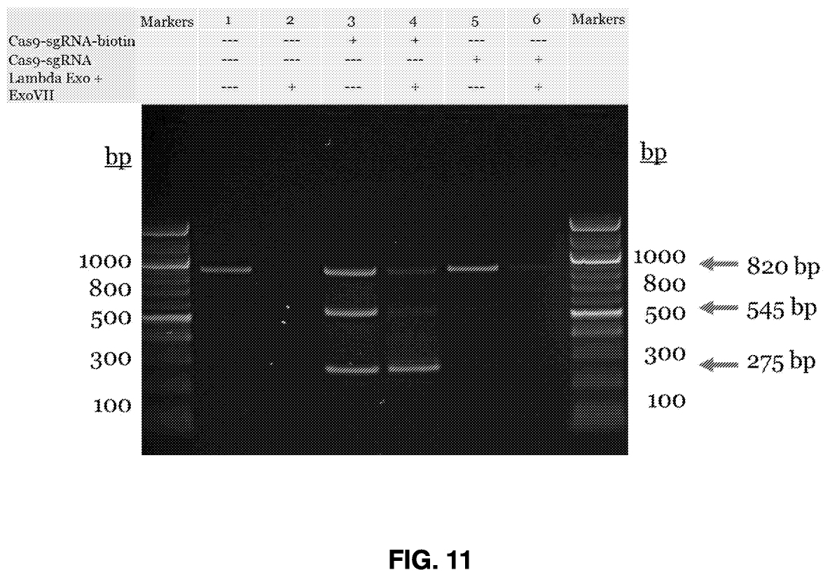

For example, as depicted in FIG. 10, the bands present in Lanes 3 and 4 at 275 bp signify that the bead Cas9-sgRNA complex with biotinylated guide RNA is able to bind to and cut the target nucleic acid in a Tris solution; Lane 4 indicates that the biotinylated complex is also able to protect the target when exposed to exonuclease; interestingly, in Lanes 3 and 4 the 275 bp band is prominent while the 545 bp band is faint. Furthermore, as depicted in FIG. 12, in human plasma for example, Lanes 3 and 4 evidence that binding and protection is strong when the biotinylated guide RNA is used on the surface of the bead; Lane 3 evidences that the amplicon is cut, but not protected as evidenced by the 545 bp band, which differs from the observation in Tris.

The results indicate that a Cas9-guide RNA complex bound to a solid surface reagent, specifically, a streptavidin coated magnetic bead is active in specifically binding target sequences, in cutting these sequences, and in protecting the targets from digestion directly in a boldly fluid sample, thereby allowing for negative enrichment directly on a solid surface.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

1. A method for enriching a sample, the method comprising:
obtaining a biological sample comprising a target nucleic acid;
binding Cas endonuclease and guide RNA that targets the target nucleic acid to a particle to form a particle complex; and
introducing the particle complex to the sample to bind to the target nucleic acid.

2. The method of claim 1, wherein the sample comprises bodily fluid.

3. The method of claim 1, wherein the particle is a magnetic material comprising magnetic, paramagnetic or superparamagnetic material.

4. The method of claim 3, wherein the magnetic material is a magnetic bead.

5. The method of claim 1, wherein the guide RNA is biotinylated.

6. The method of claim 1, further comprising introducing an exonuclease to the sample to digest unbound nucleic acid.

7. The method of claim 2, wherein the bodily fluid sample comprises bile, blood, plasma, serum, sweat, saliva, urine, feces, phlegm, mucus, sputum, tears, cerebrospinal fluid, synovial fluid, pericardia! fluid, lymphatic fluid, semen, vaginal secretion, products of lactation or menstruation, amniotic fluid, pleural fluid, rheum, or vomit.

8. The method of claim 1, wherein the target nucleic acid comprises cDNA, cfDNA, or ctDNA.

9. The method of claim 1, wherein the target nucleic acid is from a genome of a pathogen.

10. The method of claim 1, further comprising isolating the target nucleic acid.

11. The method of claim 10, wherein the isolating step comprises applying a magnetic field to separate the target nucleic acid from the particle.

12. The method of claim 10, further comprising sequencing the target nucleic acid to obtain sequence reads.

13. The method of claim 12, further comprising analyzing the sequence reads to identify one or more variants in the biological sample.

14. The method of claim 13, wherein the variant comprises a mutation specific to a tumor.

15. The method of claim 1, wherein the target nucleic acid is present at no more than about 0.01% of cell-free DNA in the bodily fluid sample.

16. The method of claim 1, further comprising detecting the target nucleic acid in the sample.

17. The method of claim 15, wherein the detection step further comprises applying a magnetic field to separate the particle complex from the sample to detect the presence of the target nucleic acid.

18. The method of claim 17, further comprising amplifying the target nucleic acid sequence.

19. A method for detecting a variant in a nucleic acid sample, the method comprising:
obtaining a biological sample comprising nucleic acid from a patient suspected of having a variant in a target nucleic acid;
enriching the sample by introducing a Cas endonuclease guide RNA particle complex to the sample to bind to the target nucleic acid, wherein the guide RNA targets the target nucleic acid;
separating the particle complex with the bound target nucleic acid from the sample; and
detecting the variant in the target nucleic acid sample.

20. The method of claim 19, wherein the detecting step comprises gel electrophoresis.
